(19) United States
(12) Patent Application Publication
(10) Pub. No.: US 2018/0110717 A1
(43) Pub. Date: Apr. 26, 2018

(54) OXIDATION DYES COMPRISING A COMBINATION OF SILICON-BLOCK COPOLYMERS AND PHOSPHOLIPIDS

(71) Applicant: Henkel AG & Co. KGaA, Duesseldorf (DE)

(72) Inventors: Sylvia Kerl, Hamburg (DE); Camille Grosjacques, Hamburg (DE)

(73) Assignee: Henkel AG & Co. KGaA, Duesseldorf (DE)

(21) Appl. No.: 15/564,738

(22) PCT Filed: Jan. 25, 2016
(86) PCT No.: PCT/EP2016/051428

§ 371 Date: Oct. 6, 2017

(30) Foreign Application Priority Data
Apr. 8, 2015 (DE) 102015206261.9

(51) Int. Cl. A61K 8/898; A61K 8/55; A61Q 5/10

(52) U.S. Cl. CPC A61K 8/898; A61K 2800/882; A61Q 5/10; A61K 8/553

(57) ABSTRACT

The disclosure relates to a cosmetic agent for dyeing keratin fibres, in particular human hair, containing a combination of a silicon-block copolymer and a phospholipid. The use of the above-mentioned combination leads to an improved chroma of hues. The invention also relates to a corresponding kit-of-parts, to the use of the cosmetic agent and the kit-of-parts and to an application method using the cosmetic agent.

### CROSS-REFERENCE TO RELATED APPLICATION

This application is a U.S. National-Stage entry under 35 U.S.C. § 371 based on International Application No. PCT/EP2016/051428, filed Jan. 25, 2016 which was published under PCT Article 21(2) and which claims priority to German Application No. 10 2015 206 261.9, filed Apr. 8, 2015, which are all hereby incorporated in their entirety by reference.

### TECHNICAL FIELD

The present disclosure relates to cosmetic agents for coloring keratin fibers, said agents containing silicone-block copolymers and phospholipids. The present disclosure also relates to a kit-of-parts, a method for coloring keratin fibers, as well as the use of cosmetic agents as contemplated herein and the kit-of-parts as contemplated herein for enhancing the colorfulness of shades.

### BACKGROUND

Changing the style and color of the hair constitutes an important area of modern cosmetics. The hair's appearance can be adapted both to current fashion trends and also to the particular preferences of each and every consumer. Coloring hair in a stylish manner or laminating graying or white hair with modern or natural color shades is normally achieved by employing color-changing agents. In addition to a strong color effect, these agents have additional features, such as increasing the hair volume.

Various coloring systems are known from the prior art for preparing color-changing cosmetic agents, more particularly for skin or for keratin fibers such as human hair, for example.

To achieve permanent, intense colors with corresponding fastness properties, so-called oxidative dyes are used. Said dyes usually contain oxidative dye products, so-called developer components and coupler components. The developer components join together or couple with one or more coupler components to form, under the influence of oxidants or atmospheric oxygen, the actual colorants per se. Indeed, the oxidative dyes are exemplified by outstanding, long-lasting color results. To achieve natural-looking colors, however, a mix from a large number of oxidative dye intermediates must normally be used; in many cases, partially-oxidizing dyes are still used to create the tinting effect.

For temporary colors, dyes or tints containing so-called partially-oxidizing agents are normally used as the coloring component. These are dye molecules that coat the substrate itself and do not require an oxidative process to create the color. These dyes include Henna, which has been known to color skin and hair since ancient times. Said dyes are usually much more sensitive to shampooing than oxidative dyes, and therefore a highly undesirable shade shift or a visible homogeneous color loss occurs at a much earlier time.

An alternative coloring method has finally aroused great interest. With this method, precursors of the natural hair dye melanin are applied to the substrate, e.g. hair; these then form, as part of the oxidative processes in the hair, natural-looking dyes. Such method typically involves the use of 5,6-dihydroxyindoline as the dye intermediate. Particularly if agents containing 5,6-dihydroxyindoline are repeatedly applied, the natural hair color of people with graying hair can be restored. The color effect can be achieved by employing atmospheric oxygen as the only oxidant, thereby eliminating the need for further oxidants. In the case of people whose hair was originally medium-blond to brown, 5,6-dihydroxyindoline can be used as the only precursor. For application on people whose hair was originally red and more particularly dark to black, on the other hand, satisfactory results can often be achieved only if other dye components, more particularly special oxidative precursors, are also used.

However, the oxidative dyes known from the prior art do not always create the desired strong color effect, more particularly a high colorfulness.

The present disclosure therefore addressed the problem of preparing a cosmetic agent for coloring keratin fibers which avoids, or at least diminishes, the disadvantages brought by the prior art and which enhances colorfulness.

### BRIEF SUMMARY

Cosmetic agent for coloring keratin fibers is provided herein. The cosmetic agent includes in a cosmetically tolerant carrier a) at least one compound, selected from the group of oxidative dye precursors, partially-oxidative dyes and the mixtures thereof. The cosmetic agent further includes b) at least one silicone block copolymer, including at least one structural unit of the formula (I) and at least one structural unit of the formula (II) and at least one structural unit of the formula (III)

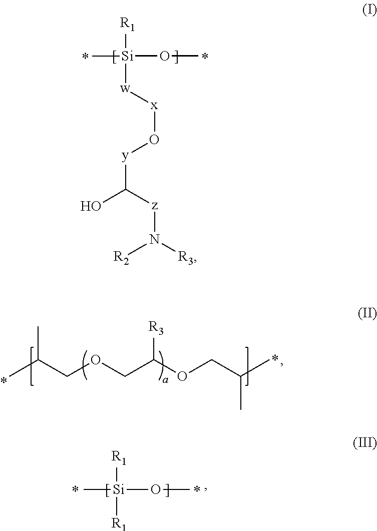

wherein
w denotes a direct bond or a heteroatom, selected from the group of NH, O or S,
x, y and z each independently denote a substituted or unsubstituted C₁-C₈-alkylene group,
R₁ denotes a substituted or unsubstituted C₁-C₄-alkyl radical,
R₂ and R₃ each independently denote a substituted or unsubstituted methyl-, ethyl-, n-propyl-, isopropyl-, n-butyl-, sec-butyl, isobutyl- or tert-butyl radical,
R₃ denotes hydrogen or a C₁-C₄-alkyl radical, and
a denotes integers from 1 to 30.
The cosmetic agent further includes c) at least one phospholipid of the formula (Phos-1)

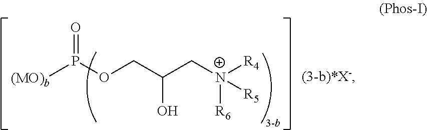

wherein
R₄ and R₆ each independently denote a C₁-C₄-alkyl radical, a C₁-C₄-hydroxyalkyl radical or a carboxyalkyl radical of the formula *—(CH2)_{c}COOM, wherein c means integers from 1 to 3 and M means hydrogen or a physiologically tolerated cation,
R₅ denotes a C₈-C₂₂-alkyl radical, a C₈-C₂₂-alkyl radical, a C₈-C₂₂-hydroxyalkyl radical or an acylaminoalkyl radical of the formula R⁷C(O)NH(CₘH₂ₘ)—*, wherein R⁷ means a linear C₈-C₂₂-acyl radical and m means an integer from 2 to 3,
B denotes integers from 0 to 3,
M denotes a physiologically tolerated cation, and
X⁻ denotes a physiologically tolerated anion.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has now emerged that the adding a combination of silicone block copolymers and phospholipids to cosmetic agents for coloring keratin fibers can enhance the colorfulness of shades. An indicator of the colorfulness of shades is the chroma. The higher the chroma value, the more colorful the shade. As contemplated herein, adding the aforementioned combination achieves a higher chroma value of the resultant hair color and therefore enhanced colorfulness.

A first subject matter of the present disclosure is therefore a cosmetic agent for coloring keratin fibers, contained in a suitable cosmetic carrier

a) at least one compound, selected from the group of oxidative dye precursors, partially-oxidative dyes and the mixtures thereof,
b) at least one silicone block copolymer, containing at least one structural unit of the formula (I) and at least one structural unit of the formula (II) and at least one structural unit of the formula (III)

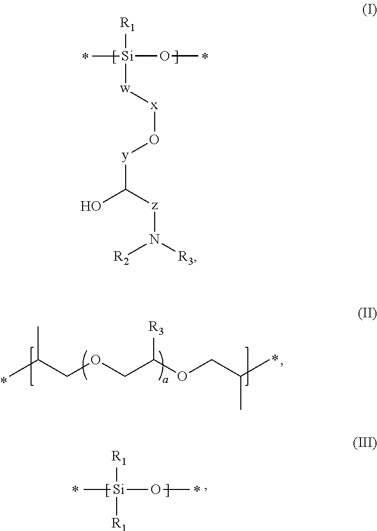

wherein
w denotes a direct bond or a heteroatom, selected from the group of NH, O or S,
x, y and z independently denote a substituted or unsubstituted C₁-C₈-alkylene group,
R₁ denotes a substituted or unsubstituted C₁-C₄-alkyl radical,
R₂ and R₃ independently denote a substituted or unsubstituted methyl-, ethyl-, n-propyl-, isopropyl-, n-butyl-, sec-butyl, isobutyl- or tert-butyl radical,
R₃ denotes hydrogen or a C₁-C₄-alkyl radical and
a denotes an integer between 1 and 30,
and
c) at least one phospholipid of the formula (Phos-I)

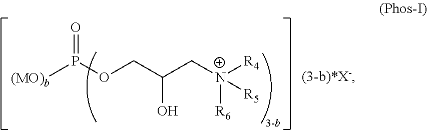

wherein
R₄ and R₆ independently denote a C₁-C₄-alkyl radical, a C₁-C₄-hydroxyalkyl radical or a carboxyalkyl radical of the formula *—(CH2)_{c}COOM, wherein c means integers from 1 to 3 and M means hydrogen or a physiologically tolerated cation,
R₅ denotes a C₈-C₂₂-alkyl radical, a C₈-C₂₂-alkyl radical, a C₈-C₂₂-hydroxyalkyl radical or an acylaminoalkyl radical of the formula R⁷C(O)NH(CₘH₂ₘ)—*, wherein R⁷ means a linear C₈-C₂₂-acyl radical and m means an integer from 2 to 3,
b denotes integers from 0 to 3,
M denotes a physiologically tolerated cation, and
X⁻ denotes a physiologically tolerated anion.

According to the above formulas, a chemical bond bearing the symbol “*” denotes a free valence of the corresponding structural fragment. A free valence is the number of atomic bonds emanating from the corresponding structural fragment at the position bearing the symbol “*”. According to the present disclosure, an atomic bond bearing the symbol preferably emanates from the structural fragments from the position bearing the symbol “*” to form other structural fragments.

As contemplated herein, the expressions “keratinic fibers and keratin fibers” suggest fur, wool, feathers as well as human hair. According to the present disclosure, a cosmetic means for coloring human hair is most preferred.

Moreover, the expression “color change” according to the present disclosure means a change to the natural hair color using dyes, which are capable of forming a color on the hair.

Additionally, the expression “block copolymer” according to the present disclosure means a block copolymer from various monomers (M1, M2, M3 etc.), wherein the polymer is formed by linking polymer blocks produced from the individual monomers, and can therefore be described by the following formula -(M1)ₓ-(M2)_{y}-(M3)_{z}-. The variables x, y and z denote the number of repeat units of the respective monomer in the corresponding polymer block.

Moreover, the expression “physiologically tolerated ions” means both anions and cations, which have no negative impact on the physical and biochemical processes of living creatures.

In addition, the expression “fatty alcohols” according to the present disclosure means aliphatic, long-chained, monovalent, primary alcohols, which have unbranched hydrocarbon radicals containing 6 to 30 carbon atoms. The hydrocarbon radicals can be either saturated or mono- or polyunsaturated.

Finally, the expression “fatty acids” according to the present disclosure means aliphatic monocarboxylic acids with unbranched carbon radicals, which have hydrocarbon radicals containing 6 to 30 carbon atoms. The hydrocarbon radicals can be either saturated or mono- or polyunsaturated.

Unless otherwise specified, the total quantity with respect to the components of the cosmetic agent as contemplated herein refers to the total quantity of active substance for the respective component. Moreover, unless otherwise specified, the total quantity with respect to the components of the cosmetic agent according to the present disclosure refers to the total weight of the oxidant-free cosmetic agent as contemplated herein.

The cosmetic agents as contemplated herein contain a cosmetically tolerant carrier. As contemplated herein, the cosmetically tolerant carrier is hydrous, alcoholic or hydrous-alcoholic. According to the present disclosure, creams, emulsions, gels or tenside-containing, foaming solutions for example, such as shampoos, foam aerosols or other preparations suitable for application on the hair, can be used.

As contemplated herein, a hydrous carrier contains at least 30 wt. %, more particularly at least 50 wt. %, of water relative to the total weight of the cosmetic agent.

According to the present disclosure, hydrous-alcoholic carriers mean aqueous compositions, containing a C₁-C₄-alcohol in a total quantity of from about 3 to about 90 wt. %, relative to the total weight of the cosmetic agent, more particularly ethanol and/or isopropanol.

The agent as contemplated herein can additionally contain other organic solvents, such as methoxybutanol, ethyldiglycol, 1,2-propylenglycol, n-propanol, n-butanol, n-butylenglycol, glycerine, diethylenglycolmonoethylether, and Diethylenglycolmono-n-butylether. All water-soluble organic solvents are preferred, wherein the solvent is contained in a total quantity of from about 0.1 to about 30 wt. %, preferably from about 1 to about 20 wt. %, more particularly from about 2 to about 10 wt. %, relative to the total weight of the cosmetic agent.

As the first essential component, the cosmetic agent as contemplated herein contains a) a compound, selected from the group of oxidative dye precursors (OFP), partially-oxidizing dyes (PO) and the mixtures thereof.

In a preferred embodiment, cosmetic agents as contemplated herein contain at least one oxidative dye precursor.

On the basis of their reaction behavior, oxidative dye precursors can be divided into two categories, so-called developer components and coupler components. Developer components can combine together to form the actual dye. The cosmetic agent as contemplated herein can therefore contain these components as the sole compounds. In a preferred embodiment, the cosmetic agents as contemplated herein therefore contain at last one oxidative dye precursor of the developer type. Within the scope of the present disclosure, however, it may be that the cosmetic agent as contemplated herein contains at least one oxidative dye precursor of the coupler type. Particularly good results with respect to the coloration of keratin fibers are achieved when the cosmetic agents as contemplated herein contain at least one oxidative dye precursor of the developer type and at least one oxidative dye precursor product of the coupler type.

The developer and coupler components are normally used in a free form. In the case of substances with amino groups, however, use of the salt form thereof, more particularly in the form of hydrochlorides and hydrobromides or sulfates, may be preferred.

As contemplated herein, the preference is for cosmetic agents which contain the developer and/or coupler components, each in a total quantity of from about 0.001 to about 10 wt. %, preferably from about 0.01 to about 8 wt. %, more preferably from about 0.1 to about 5 wt. %, most preferably from about 0.5 to about 3 wt. %, relative to the total weight of the cosmetic agent.

In another preferred embodiment, the cosmetic agent as contemplated herein contains an oxidative dye precursor of the developer and/or coupler components in a total quantity of from about 0.001 to about 10 wt. %, preferably from about 0.01 to about 8 wt. %, more preferably from about 0.1 to about 5 wt. %, most preferably from about 0.5 to about 3 wt. %, relative to the total weight of the cosmetic agent.

Suitable oxidative dye precursors of the developer type are typically p-phenylendiamine and the derivatives thereof. Preferred p-phenylendiamines are selected from one or multiple compounds of the group, which is formed from p-phenylendiaminr, p-toluylendiamind, 2-chlor-p-phenylendiaminr, 2,3-fimethyl-p-phenylendiaminr, 2,6-fimethyl-p-phenylendiamine, N,N-bis-(2-hydroxyethyl)-p-phenylen-diamine, 2-(2-hydroxyethyl)-p-phenylendiamine, 2-(1,2-dihydroxyethyl)-p-phenylendiamine, N-(2-hydroxypropyl)-p-phenylendiamine, N-(4′-aminophenyl)-p-phenylendiamine, 2-methoxymethyl-p-phenylendiamine, N-phenyl-p-phenylendiamine, 2-(2-hydroxyethyloxy)-p-phenylendiamine and N-(4-amino-3-methyl-phenyo)-N-[3-(1H-imidazol-1-yl)propyl]amine, as well as the physiologically-tolerable salts thereof.

As contemplated herein, use of compounds containing at least two aromatic rings, which are substituted with amino and/or hydroxyl groups, as the developer components may be preferred. Preferred two-ring developer components are selected from N,N′-bis-(2-hydroxyethyl)-N,N′-bis-(4′-aminophenyl)-1,3-diaminopropan-2-ol, N,N′-bis-(4-aminophenyl)-1,4-diazacycloheptane, bis-(2-hydroxy-5-aminophenyl)methane, as well as the physiologically-tolerable salts thereof.

As contemplated herein, use of a p-aminophenol derivative or one of the physiologically-tolerable salts thereof as the developer component may also be preferred. Preferred p-aminophenols are p-aminophenol, N-methyl-p-aminophenol, 4-amino-3-methylphenol, 4-amino-2-aminomethyl-phenol, 4-amino-2-(1,2-dihydroxyethyl)phenol, 4-amino-2-(diethylaminomethyl)phenol, as well as the physiologically-tolerable salts thereof. The developer components can also be selected from o-aminophenol and the derivatives thereof, preferred from 2-amino-4-methylphenol, 2-amino-5-methylphenol, 2-amino-4-chlorophenol and/or the physiologically-tolerable salts thereof.

Moreover, the developer components can be selected from heterocyclic developer components, such as pyrimidine derivatives, pyrazole derivatives, pyrazolopyrimidine derivatives and/or the physiologically-tolerable salts thereof. Preferred pyrimidine derivatives are 2,4,5,6-tetraaminopyrimidine, 4-hydroxy-2,5,6-triaminopyrimidine and the physiologically-tolerable salts thereof. A preferred pyrazole derivative is 4,5-diamino-1-(2-hydroxyethyl)pyrazole, as well as the physiologically-tolerable salts thereof. Pyrazolo[1,5-a]pyrimidine are particularly preferred as pyrazolopyrimidines.

Preferred oxidative dye precursors of the developer type are selected from the group formed from p-phenylendiamine, p-toluylenediamine, 2-(2-hydroxyethyl)-p-phenylendiamine, 2-(1,2-dihydroxyethyl)-p-phenylendiamine, N,N-bis-(2-hydroxyethyl)-p-phenylendiamine, 2-methoxymethyl-p-phenylendiamine, N-(4-amino-3-methylphenyl)-N-[3-(1H-imidazol-1-yl) propyl]amine, N,N′-bis-(2-hydroxy-ethyl)-N,N′-bis-(4-aminophenyl)-1,3-diamino-propan-2-ol, bis-(2-hydroxy-5-aminophenyl)methane, 1,3-bis-(2,5-diaminophenoxy)-propan-2-ol, N,N′-bis-(4-aminophenyl)-1,4-diazacycloheptane, 1,10-bis-(2,5-diaminophenyl)-1,4,7,10-tetraoxadecan,

p-aminophenol, 4-amino-3-methylphenol, 4-amino-2-amino-methylphenol, 4-amino-2-(1,2-dihydroxyethyl)phenol and 4-amino-2-(diethylaminomethyl)phenol, 4,5-amino-1-(2-hydroxyethyl)pyrazole, 2,4,5,6-tetraamino-pyrimidine, 4-hydroxy-2,5,6-triaminopyrimidine, 2-hydroxy-4,5,6-triaminopyrimidine or the physiologically-tolerable salts thereof.

Particularly preferred developer components are p-toluylenediamine, 2-(2-hydroxyethyl)-p-phenylen-diamine, 2-methoxymethyl-p-phenylendiamine, N-(4-amino-3-methylphenyl)-N-[3-(1-H-imidazol-1-yl)-propyl)amine, and/or 4,5-diamino-1-(2-hydroxyethyl)pyrazole, as well as the physiologically-tolerable salts thereof.

According to another preferred embodiment of the present disclosure, the cosmetic agent as contemplated herein contains, as oxidative dye precursors, at least one coupler component in addition to at least one developer component. m-phenylendiaminderivatives, naphthols, resorcin and resorcin derivatives, pyrazolones and m-aminophenolderivatives are typically used as coupler components.

Preferred coupler components as contemplated herein are selected from

(A) m-aminophenol and the derivatives thereof, more particularly 3-aminophenol, 5-amino-2-methylphenol, 3-amino-2-chlor-6-methylphenol, 5-amino-4-chlor-2-methylphenol, 5-(2′-hydroxyethyl)-amino-2-methylphenol and 2,4-dichlor-3-aminophenol,
(B) o-aminophenol and the derivatives thereof, such as 2-amino-5-ethylphenol,
(C) m-Diaminobenzol and the derivatives thereof, such as 2,4-diaminophenoxy-ethanol, 1,3-bis-(2′,4′-diaminophenoxy)-propane, 1-methoxy-2-amino-4-(2′-hydroxyethylamino) benzol, 2,6-bis-(2′-hydroxyethylamino)-1-methylbenzol, 2-({3-[(2-Hydroxyethyl)amino]-4-methoxy-5-methylphenyl}amino)ethanol and 2-({3-[(2-hydroxyethyl)amino]-2-methoxy-5-methylphenyl}-amino)ethanol,
(D) o-diaminobenzol and the derivatives thereof,
(E) di and/or trihydroxybenzol derivatives, more particularly resorcin, 2-chlorresorcin, 4-chlorresorcin, 2-methylresorcin and 1,2,4-trihydroxybenzol,
(F) pyridine derivatives, more particularly 3-amino-2-methylamino-6-methoxypyridine, 2,6-diamino pyridine, 2,6-dihydroxy-3,4-dimethylpyridine, 2-amino-3-hydroxypyridine and 3,5-diamino-2,6-dimethoxy-pyridine,
(G) naphthaline derivatives, such as 1-naphthol and 2-methyl-1-naphthol,
(H) morpholine derivatives, such as 6-hydroxybenzomorpholine,
(I) quinolaxine derivatives,
(J) pyrazol derivatives, such as 1-phenyl-3-methylpyrazol-5-on,
(K) indol derivatives, such as 6-hydroxyindol,
(L) pyrimidine derivatives or
(M) methylendioxybenzol derivatives, such as 1-(2′-Hydroxyethyl)-amino-3,4-methylendioxybenzol, as well as the physiologically-tolerable salts thereof.

Coupler components preferred as contemplated herein are selected from the group, which is formed from 3-aminophenol, 5-amino-2-methylphenol, 3-amino-2-chlor-6-methylphenol, 2-hydroxy-4-aminophenoxyethanol, 5-amino-4-chlor-2-methylphenol, 5-(2-hydroxyethyl)-amino-2-methylphenol, 2,4-dichlor-3-aminophenol, 2-aminophenol, 3-phenylendiamine, 2-(2,4-diaminophenoxy)ethanol, 1,3-bis(2,4-diaminophenoxy)propane, 1-methoxy-2-amino-4-(2-hydroxyethylamino)benzol, 1,3-bis(2,4-diaminophenyl)propane, 2,6-bis(2′-hydroxyethylamino)-1-methylbenzol, 2-({3-[(2-hydroxyethyl)amino]-4-methoxy-5-methylphenyl}amino)ethanol, 2-({3-[(2-hydroxyethyl)amino]-2-methoxy-5-methylphenyl)amino)ethanol, 2-({3-[(2-hydroxyethyl)amino]-4, 5-dimethylphenyl}amino)ethanol, 2-[3-morpholine-4-ylphenyl)amino]ethanol, 3-amino-4-(2-methoxy-ethoxy)-5-methylphenylamine, 1-amino-3-bis-(2-hydroxyethyl)aminobenzol, resorcin, 2-methylresorcin, 4-chlorresorcin, 1,2,4-trihydroxybenzol, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-on, 1-naphthol, 1,5-dihydroxynaphthaline, 2,7-dihydroxynaphthaline, 1,7-dihydroxynaphthaline, 1,8-dihydroxynaphthaline, 4-hydroxyindole, 6-hydroxyindole, 7-hydroxyindole, 4-hydroxyindoline, 6-hydroxyindoline, 7-hydroxyindoline or the physiologically-tolerable salts of the aforementioned compounds.

Preferred coupler components as contemplated herein are resorcin, 2-methylresorcin, 5-amino-2-methylphenol, 3-aminophenol, 2-(2,4-diaminophenoxy)ethanol, 1,3-bis-(2,4-diamino-phenoxy)propane, 1-methoxy-2-amino-4-(2′-hydroxyethylamino)benzol, 2-amino-3-hydroxypyridine, 2,6-dihydroxy-3,4-dimethylpyridine, 1,5-dihydroxynaphthaline, 2,7-dihydroxynaphthaline and 1-naphthol, as well as the physiologically-tolerable salts thereof.

In a particularly preferred embodiment of the present disclosure, the cosmetic agents as contemplated herein are exemplified in that they contain at least one developer component, selected from the group of p-phenylendiamine, p-toluylendiamine, N,N-bis-(2-hydroxyethyl)amino-p-phenylendiamine, 1,3-bis-[(2-hydroxyethyl-4′-aminophenyl)amino]-propan-2-ol, 1,10-bis-(2′,5′-diaminophenyl)-1,4,7,10-tetraoxadecane 4-aminophenol, 4-amino-3-methylphenole, bis-(5-amino-2-hydroxyphenyl)methane, 2,4,5,6-tetraaminopyrimidine, 2-hydroxy-4, 5,6-triaminopyrimidine, 4,5-diamino-1-(2-hydroxyethyl)-pyrazole, the physiologically-tolerable salts thereof and the mixtures thereof, and at least one coupler component, selected from the group of resorcin, 2-methylresorcin, 5-methylresorcin, 2,5-dimethylresorcin, 4-clorresorcin, resorcin monomethylether, 5-aminophenole, 5-amino-2-methylphenole 5-2-hydroxyethyl)amino-2-methylphenol, 3-amino-4-chlor-2-methylphenole, 3-amino-2-chlor-6-methylphenole, 3-amino-2,4-dichlorphenole, 2,4-diaminophenoxyethanol, 2-amino-4-(2′-hydroxyethyl)amino-anisolsulfate, 1,3-bis-(2,4-diaminophenoxy)propane, 2-amino-3-hydroxypyridine, 2-methylamino-3-amino-6-methoxypyridine, 2,6-dihydroxy-3,4-dimethylpyridine, 3,5-diamino-2,6-dimethoxypyridine, 1-naphthol, 2-methyl-1-naphthol, 1,5-dihydroxynaphthaline, 2,7-dihydroxynaphthaline, 1-phenyl-3-methylpyrazol-5-on, 2,6-bis-[(2′-hydroxyethyl)amino]-toluol, 4-hydroxyindol, 6-hydroxyindol, 6-hydroxybenzomorpholine, the physiologically-tolerable salts thereof and the mixtures thereof.

In order to obtain a balanced and suitable shade formation, the present disclosure may specify that the cosmetic agents as contemplated herein additionally contain at least one partially-oxidizing dye. Partially-oxidizing dyes are dyes that that coat the substrate itself and do not require an oxidative process to create the color. Partially-oxidizing dyes are usually nitro-phenylendiamines, nitroaminophenols, azo dyes, anthrachinones or indophenols.

Partially-oxidizing dyes can be divided between anionic, cationic and non-ionic partially-oxidizing dyes.

Preferred anionic partially-oxidizing dyes are the compounds known under the designations Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52 and tetrabromphenole blue. Preferred cationic partially-oxidizing dyes are cationic triphenylmethane dyes, such as Basic Blue 7, Basic Blue 26, BasicVialet 2 and Basic Violet 14, as well as aromatic systems, which are substituted with a quarternary nitrogen group, such as Basic Yellow 57, Basic Red 76, Basic Blue 99, Basic Brown 16 and Basic Brown 17 and HC Blue 16, as well as Basic Yellow 87, Basic Orange 31 and Basic Red 51. Preferred non-ionic partially-oxidizing dyes are HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzol, 2-amino-4-nitrophenole, 1,4-bis-(2-hydroxyethyl)amino-2-nitrobenzol, 3-nitro-4-(2-hydroxyethyl)aminophenole, 2-(2-hydroxyethyl)amino-4,6-d initrophenole, 4-[(2-hydroxyethyl)amino]-3-nitro-1-methylbenzol, 1-amino-4-(2-hydroxyethyl)amino-5-chlor-2-nitrobenzol, 4-amino-3-nitrophenol, 1-(2′-ureidoethyl)amino-4-nitrobenzol, 2-[(4-Amino-2-nitrophenyl)amino]benzoic acid, 6-nitro-1,2,3,4-tetrahydrochinoxaline, 2-hydroxy-1,4-naphthochinon, picramic acid and the salts thereof, 2-amino-6-chloro-4-nitrophenol, 4-ethylamino-3-nitrobenzoic acid and 2-chlor-6-ethylamino-4-nitrophenol.

Moreover, partially-oxidizing dyes that occur in nature, such as Henna red, Henna neutral, Henna black, chamomile blossoms, sandalwood, black tea, walnut, Cascara bark, sage, logwood, madder root, catechu, ceder and alkanna root, can also be used.

As contemplated herein, the preference is for cosmetic agents which contain the partially-oxidizing agent in a total quantity of from about 0.001 to about 10 wt. %, preferably from about 0.01 to about 8 wt. %, more preferably from about 0.1 to about 5 wt. %, most preferably from about 0.5 to about 3 wt. %, relative to the total weight of the cosmetic agent.

The cosmetic agents as contemplated herein contain as a second essential component b) at least one special silicone block copolymer. The addition of this special silicone block copolymer in conjunction with a phospholipid leads to enhanced colorfulness of shades, and therefore a smaller quantity of dyes and/or dye precursors, such as developer components, coupler components, partially-oxidizing dyes, as well as the mixtures thereof, must be used in order to achieve an identical colorfulness to that obtained when using cosmetic agents without silicone block copolymers and phospholipids.

In the formula (I), preferably w denotes a direct bond or an oxygen atom, x for a C₂-C₄-alkyl group, y and z, independently denote a C₁-C₃-alkyl group, and R₁ and R₂, independently denote a hydroxypropyl radical.

It has proven advantageous according to the present disclosure if, in the formula (II), radical R₃ denotes a hydrogen atom and a denotes an integer from 4 to 25, preferably from 6 to 20, preferably from 8 to 18, more preferably from 12 to 16.

As contemplated herein, the silicone block copolymer more preferably contains b) at least one structural unit (Ia)

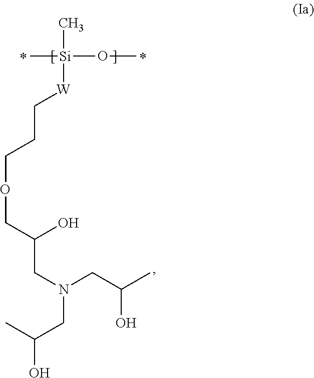

wherein
w denotes a direct bond or an oxygen atom.

As contemplated herein, particularly good results are achieved if the cosmetic agent contains, as a silicone block copolymer b) silicone block copolymer of the formula (IV)

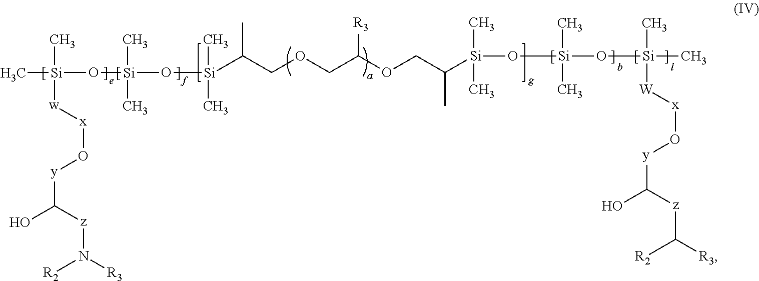

wherein
w denotes a direct bond or an oxygen atom,
x denotes a propyl group,
y and z each independently denote a methyl group,
R₂ and R₃ each independently denote a 2-hydroxypropyl radical,
R₃ denotes hydrogen, and
a denotes integers from 4 to 25, preferably from 6 to 20, more preferably from 8 to 18, and most preferably from 12 to 16.

The use of silicone block copolymers of the formula (IV) in combination with a phospholipid results in enhanced colorfulness of shades. As a result, the quantity of used dyes can be significantly less than the quantity of cosmetic agents not containing the aforementioned combination, without, however, negatively impacting the colorfulness of the corresponding shades.

As contemplated herein, it is particularly advantageous if the at least one silicone block copolymer b) is a silicone block copolymer from polyethylenglycol-bis(2-methyl-2-propen-1-yl)ether, 3-[3-[bis(2-hydroxypropyl)amino]-2-hydroxypropoxy]propyl-siloxane and dimetyhlsiloxane. The use of such silicone block copolymers in combination with phospholipids achieves particularly good results with respect to the enhanced colorfulness of shades.

A particularly preferred silicone block copolymer according to this embodiment is a block copolymer with the INCI designation bis-diisopropanolamino-PG-propyl dimethicone/bis-isobutyl PEG-14 copolymer (CAS Number 1253692-80-6). Such block copolymers are distributed, for example, under the trading name Dow Corning CE-8411 Smooth Emulsion Plus by the company Dow Corning.

The at least one silicone block copolymer b) is contained in the cosmetic agents as contemplated herein in a total quantity of from about 0.0001 to about 20 wt. %, preferably from about 0.0005 to about 10 wt.-%, more preferably from about 0.005 to about 8.0 wt. %, even more preferably from about 0.01 to about 5.0 wt. %, and most preferably from about 0.1 to about 1.0 wt. %, relative to the total weight of the cosmetic agent. The use of the aforementioned total quantity of the at least one special silicone block copolymer leads, in combination with the phospholipid, to an enhanced colorfulness of shades. Moreover, the aforementioned quantity of the special silicone block copolymer effectively prevents any incompatibility with other ingredients of the cosmetic agent as contemplated herein, and therefore a high storage stability of the cosmetic agent is guaranteed when said quantities are used.

The cosmetic agents as contemplated herein contain as a third essential component c) at least one special phospholipid. The addition of this special phospholipid in conjunction with the aforementioned silicone block copolymer leads to enhanced colorfulness of shades, and therefore a smaller quantity of dyes and/or dye precursors, such as developer components, coupler components, partially-oxidizing dyes, as well as the mixtures thereof, must be used in order to achieve an identical colorfulness to that obtained when using cosmetic agents without phospholipids and silicone block copolymers.

It has proven advantageous according to the present disclosure if, in the formula (Phos-1), radical R₅ denotes an acylaminoalkyl radical of the formula R⁷C(O)NH(CₘH₂ₘ)—*, wherein R⁷ means a linear C₈-C₂₂-acyl radical and m denotes an integer from 2 to 3, radicals R₄ and R₆, each independently denote a C₁-C₂-alkyl radical, b denotes the integer 0 or 1, M denotes an alkali metal, more particularly lithium, potassium or sodium, and X⁻ denotes a halide ion, more particularly chloride, bromide or iodide.

Particularly good results with respect to the enhanced colorfulness of shades are achieved when phospholipids of the formula (Phos-1a) are used in combination with the aforementioned silicone block copolymers. A particularly preferred embodiment is for the cosmetic agent to contain, as the phospholipid c) a phospholipid of the formula (Phos-1a)

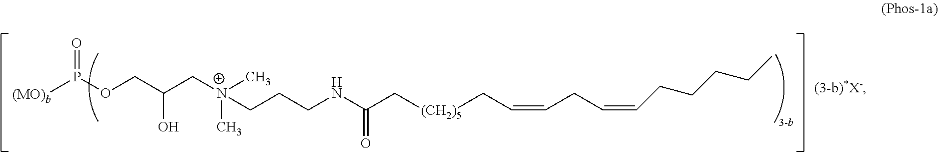

wherein
M denotes a sodium ion
X⁻ denotes a chloride ion, and
b denotes the integer 0 or 1.

The use of phospholipids of the formula (Phos-1a) in combination with the aforementioned silicone block copolymers, more particularly with the silicone block copolymers of the formula (IV), results in an improved colorfulness of shades. As a result, the quantity of used dyes can be significantly less than the quantity of cosmetic agents not containing the aforementioned combination, without, however, negatively impacting the colorfulness of the corresponding shades.

A most preferred phospholipid according to this embodiment is a phospholipid with the INCI designation linoleamidopropyl PG-dimonium chloride phosphate. Said phospholipids are distributed, for example, under the trade name Arlasilk phospholipid EFA by the company Croda.

The at least one phospholipid c) is contained in the cosmetic agents as contemplated herein in a total quantity of from about 0.0001 to about 10 wt. %, preferably from about 0.0005 to about 5.0 wt.-%, more preferably from about 0.005 to about 2.0 wt. %, even more preferably from about 0.001 to about 1.0 wt. %, and most preferably from about 0.05 to about 0.5 wt. %, relative to the total weight of the cosmetic agent. The use of the aforementioned total quantity of the at least one special phospholipid leads, in combination with the aforementioned silicone block copolymer phospholipid, to an enhanced colorfulness of shades. Moreover, the aforementioned quantity of the special phospholipids effectively prevents any incompatibility with other ingredients of the cosmetic agent as contemplated herein, and therefore a high storage stability of the cosmetic agent is guaranteed when said quantities are used.

It has emerged that an adding cyclosiloxanes is able to stabilize the at least one silicone block copolymer, more particularly the silicone block copolymer of the formula (IV), in the cosmetic agents as contemplated herein, thereby significantly enhancing the colorfulness of the shades. Therefore, preferred coupler components as contemplated herein additionally contain dimethylcyclosiloxane in a total quantity of less than 1 wt. %, relative to the total weight of the cosmetic agent, wherein the dimethylcyclosiloxane has the formula (V)

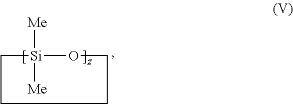

wherein
z denotes integers from 2 to 10, preferably from 4 to 10, more preferably 6, 7 or 8.

The cosmetic agents as contemplated herein can contain other active ingredients and additives. According to the present disclosure, it is therefore preferable for the cosmetic agent to contain in addition at least one other compound, selected from the group of (i) thickening agents; (ii) linear or branched, saturated or unsaturated alcohols with 8 to 20 carbon atoms; (iii) tensides, more particularly amphoteric tenside; (iv) alkalizing agents; (v) oils; as well as (vi) the mixtures thereof.

Preferably, the cosmetic means as contemplated herein are formulated as free-flowing preparations. The cosmetic agents must be formulated in such a manner that they can be readily applied and distributed at the place of use on the one hand, but on the other are adequately viscous such that they remain at the site of action and do not run during the exposure time.

It has proven advantageous according to the present disclosure for the cosmetic agent as contemplated herein to contain at least one thickening agent from the group of (i) anionic, synthetic polymers; (ii) cationic, synthetic polymers; (iii) naturally occurring thickening agents, such as non-ionic guargums, scleroglucangums or xanthangums, rummi arabicum, Ghatti rubber, Karaya rubber, Tragant rubber, Carrageen rubber, Agar-Agar, locust bean gum, pectines, alginates, starch fractions and derivatives, such as amylose, amylopectin and dextrines, as well as cellulose derivatives, such as methyl cellulose, carboxyalkyl celluloses and hydroxyalkyl celluloses; (iv) non-ionic synthetic polymers, such as polyvinyl alcohol or polyvinylpyrrolidinon; (v) inorganic thickening agents, more particularly phyllosilicates, such as bentonit, more particularly smektites, such as montmorillonite or hectorit; as well as (vi) the mixtures thereof, in a total quantity of from about 0.0005 to about 5.0 wt. %, preferably from about 0.001 to about 3.0 wt. %, more preferably from about 0.005 to about 1.0 wt. %, most preferably from about 0.008 to about 0.01 wt. %, relative to the total weight of the cosmetic agent.

It has proven advantageous according to the present disclosure for the cosmetic agent as contemplated herein to contain, as the thickening agent, at least one naturally occurring thickening agent, more particularly Xanthangum, as well as the salts thereof, of from about 0.0005 to about 5.0 wt. %, preferably from about 0.001 to about 1.0 wt. %, more preferably from 0.005 to about 1.0 wt. %, most preferably from about 0.01 to about 0.1 wt. %, relative to the total weight of the cosmetic agent.

According to the present disclosure, it can be preferable for the linear or branched, saturated or unsaturated alcohol with 8 or 20 carbon atoms to be selected from the group of myristyl alcohol (1-tetradecanol), stearyl alcohol (1-octadecanol), cetearyl alcohol, 2-octyldodecanol, arachyl alcohol (eicosan-1-ol), gadoleyl alcohol ((9Z)-eicos-9-en-1-ol), arachidon alcohol ((5Z,8Z,11Z,14Z)-eicosa-5,8, 11,14-tetraen-1-ol), preferably 2-octyldodecanol and/or cetearyl alcohol, and in a total quantity of from about 1.0 to about 35 wt. %, preferably from about 5.0 to about 30 wt. %, more preferably from about 10 to about 25 wt. %, most preferably from about 12 to about 20 wt. %, relative to the total weight of the cosmetic agent.

Preferably, the cosmetic agents as contemplated herein can also contain at least one partial ester from a polyol with 2 to 6 carbon atoms and linear saturated carbon atoms with 12 to 30, more particularly 14 to 22 carbon atoms, wherein the partial ester can be hydroxylated, in a total quantity of 0.5 to 10 wt. %, more particularly 3.0 to 8.0 wt. %, relative to the total weight of the cosmetic agent. Said partial esters are more particularly the mono and diesters of glycerine or the monoesters of propylenglycol or the mono and diesters of ethylenglycol or the mono, di, tri and tetraesters of pentaerythrit, each with linear saturated C₁₂-C₃₀ carboxylic acids, which can be hydroxylated, more particularly those with palmitic and stearic acids, the sorbitanmono-, -di- or -triesters of linear saturated C₁₂-C₃₀ carboxylic acids, which can be hydroxylated, more particularly those of myristin acids, palmitic acids, stearic acids or of mixtures of said fatty acids and the methylglucosemono- and diesters of linear saturated C₁₂-C₃₀ carboxylic acids, which can be hydroxylated.

According to the present disclosure, the cosmetic agent as contemplated herein can contain at least one polyolpartial ester, selected from glycerine monostearate, glycerin monopalmitate, glycerin distearate, glycerin dipalmitate, ethylenglycol monostearate, ethylenglycolmono palmitate, ethylenglycol distearate, ethylenglycoldipalmitate, as well as the mixtures thereof, more particularly mixtures from glycerinmonostearate, glycerinmonopalmitate, glycerindistearate and glycerindipalmitate in a total quantity of from about 0.5 to about 10 wt. %, more particularly from about 3.0 to about 8.0 wt. %, relative to the total weight of the cosmetic agent.

The use of the aforementioned alcohols, partial esters and polypartial esters in the cosmetic agent as contemplated herein can be particularly preferred if the cosmetic agent as contemplated herein exists in the form of an oil-in-water emulsion.

According to the present disclosure, the cosmetic agent as contemplated herein can also contain at least one tenside. Tensides according to the present disclosure are amphiphilic (bifunctional) compounds, which consist of at least one hydrophobic and at least one hydrophilic molecular part. A basic property of tensides and emulsifiers is the oriented absorption at boundary surfaces, as well as the aggregation to micelles and the formation of lyotrophic phases.

According to a preferred embodiment of the present disclosure, the cosmetic agents as contemplated herein contain at least one amphoteric tenside in a total quantity of from about 0.1 to about 5.0 wt. %, more particularly from about 0.2 to about 2.0 wt. %, relative to the total weight of the cosmetic agent. Amphoteric and/or zwitterionic tensides are surface active compounds, which have at least one quarternary ammonium group and at least one —COO⁽⁻⁾- or —SO3⁽⁻⁾-group.

According to the present disclosure, the compounds below are particularly preferred amphoteric tensides:
Alkylbetains with 8 to 20 carbon atoms in the alkyl group,
Amidopropylbetaines with 8 to 20 carbon atoms in the acyl group,
sulfobetains with 8 to 20 carbon atoms in the acyl group and
amphoacetates or amphodiacetates with 8 to 20 carbon atoms in the acyl group.

In a particularly preferred embodiment, the cosmetic agents as contemplated herein contain as tenside at least one amphoteric tenside, selected from amidopropylbetaines with 9 to 13 carbon atoms in the acyl group, in a total quantity of from about 0.1 to about 5.0 wt. %, more particularly from about 0.2 to about 2.0 wt. %, relative to the total weight of the cosmetic agent.

The cosmetic agents as contemplated herein can also contain at least one ethoxylated non-ionic tenside in a total quantity of from about 0.5 to about 6.0 wt. %, more particularly from about 1.0 to about 4.0 wt. %, relative to the total weight of the cosmetic agent. The ethoxylated non-ionic tensides are different from the previously mentioned ethoxylated primary alcohols. It has proven particularly advantageous for the ethoxylated non-ionic tenside to have a HLB value of above 10, preferably above 13. This requires the non-ionic tenside to have an adequately high degree of ethoxylation. In this context, the cosmetic agent as contemplated herein therefore contains, as the ethoxylated, non-ionic tenside, at least one ethoxylated tenside with at least 12 ethylenoxide units. In addition to the correspondingly ethoxylated fatty alcohols, more particularly lauryl alcohol, myristyl alcohol, getyl alcohol, stearyl alcohol, arachylalcohol and behenyl alcohol, the present disclosure states that the sediments of from about 20 to about 60 mol of ethylenoxide for castor oil and hydrogenated castor oil are particularly suitable. The at least one ethoxylated non-ionic tenside is preferably selected from tensides with the INGI designation geteth-12, steareth-12, geteareth-12, geteth-20, steareth-20, geteareth-20, geteth-30, steareth-30, getea-reth-30, oleth-30, geteareth-50, PEG-40 hydrogenated castor oil and PEG-60 hydrogenated castor oil, as well as mixtures of said substances, more preferably selected from geteth-20, steareth-20, geteareth-20, geteth-30, steareth-30 and geteareth-30.

Cosmetic agents according to the present disclosure usually have an alkaline pH value, more particularly between about pH 8.0 and about pH 12. These pH values are required to guarantee an opening of the external cuticle layer (cuticula) and facilitate a penetration of oxidative dye precursors and/or of the oxidant into the hair.

The aforementioned pH value can preferably be set using an alkalizing agent. According to the present disclosure, the alkalizing agent is selected from the group of (i) inorganic alkalizing agents; (ii) organic alkalizing agents; as well as (iii) the mixtures thereof, and in a total quantity of from about 1.5 to about 9.5 wt. %, preferably from about 2.5 to about 8.5 wt. %, more preferably from about 3.0 to about 8.0 wt. %, most preferably from about 3.5 to about 7.5 wt. %, relative to the total weight of the cosmetic agent.

Preferred inorganic alkalizing agents are selected from the group formed from ammoniac and/or ammonium hydroxide, i.e. hydrous solutions of ammoniac, sodium hydroxide, potassium hydroxide, calcium hydroxide, barium hydroxide, sodium phosphate, potassium phosphate, sodium silicate, potassium silicate, sodium carbonate and potassium carbonate, as well as mixtures thereof. Ammonia and/or ammonium hydroxide is a most preferred alkalizing agent. Ammonia is most preferable in a total quantity of from about 0.1 to about 20 wt. %, preferably from about 0.5 to about 10 wt. %, more particularly from about 1.0 to about 7.0 wt. %, relative to the total weight of the cosmetic agent.

Preferred organic alkalizing agents are selected from at least one alkanolamine. Alkanolamines preferred as contemplated herein are selected from alkanolamines from primary, secondary or tertiary amines with a C₂-C₆ alkyl base body, which carries at least one hydroxyl group. Particularly preferred alkanalamines are selected from the group formed from 2-aminoethan-1-ol (monoethanolamine), 3-aminopropan-1-ol, 4-amino-butan-1-ol, 5-aminopentan-1-ol, 1-aminopropan-2-ol (monoisopropanolamine), 1-aminobutan-2-ol, 1-aminopentan-2-ol, 1-aminopentan-3-ol, 1-aminopentan-4-ol, 2-amino-2-methyl-propanol, 2-amino-2-methylbutanol, 3-amino-2-methylpropan-1-ol, 1-amino-2-methylpropan-2-ol, 3-aminopropan-1,2-diol, 2-amino-2-methylpropan-1,3-diol, 2-amino-2-ethyl-1,3-propandiol, N,N-dimethyl-ethanolamine, triethanolamine, diethanolamine and triisopropanolamine. Most preferred alkanalamines as contemplated herein are selected from the group of 2-aminoethan-1-ol (monoethanolamine), 2-amino-2-methylpropan-1-ol, 2-amino-2-methyl-propan-1,3-diol and triethanolamine. Particularly preferred cosmetic agents as contemplated herein contain a mixture of Mono-ethanolamine and 2-amino-2-methylpropan-1ol. It is preferable for the at least one alkanalamine to be contained in a total quantity of from about 0.05 to about 15 wt. %, more preferably from about 0.5 to about 10 wt. %, most preferably from about 3.5 to about 7.5 wt. %, relative to the total weight of the cosmetic agent.

Other preferred organic alkalizing agents as contemplated herein are selected from alkaline amino acids, particularly preferred from the group formed from L-arginine, D-arginine, D/L-arginine, L-lysine, D-lysine, D/L-lysine, as well as the mixtures thereof. Most preferred alkaline amino acids as contemplated herein are selected from L-arginine, D-arginine and D/L-arginine. Preferred cosmetic agents as contemplated herein contain at least one alkalizing agent that differs from alkanalamines and ammoniac in a total quantity of from about 0.05 to about 5.0 wt. %, more particularly from about 0.5 to about 3.0 wt. %, relative to the total weight of the cosmetic agent.

In a particularly preferred embodiment, the cosmetic agents as contemplated herein contain a mixture of at least two different alkalizing agents, more particularly monoethanolamine and ammonia, in a total quantity of from about 0.05 to about 15 wt. %, preferably from about 0.5 to about 10 wt. %, preferably from about 3.5 to about 7.5 wt. %, relative to the total weight of the cosmetic agent.

Preferably, the pH value of the cosmetic agent as contemplated herein, measured at about 22° C., is from about 8 to about 13, preferably from about 9.5 to about 12, more preferably from about 10 to about 11.5, most preferably from about 10.5 to about 11.

According to the present disclosure, it can also be preferable for the cosmetic agent to contain at least one oil, selected from the group of sunflower oil, corn oil, soya oil, pumpkin seed oil, grape seed oil, sesame oil, hazelnut oil, apricot seed oil, macadamia nut oil, arara oil, castor oil, avocado oil, as well as the mixtures thereof, in a total quantity of from about 0.1 to about 10 wt. %, preferably from about 0.2 to about 5.0 wt. %, more particularly from about 0.5 to about 2.0 wt. %, relative to the total weight of the cosmetic agent. The use of at least one of the aforementioned oils can significantly increase the nourishing effect of the non-ionic linear silicone polymers.

The cosmetic agent as contemplated herein most preferably contains grape seed oil in a total quantity of from about 0.1 to about 10 wt. %, preferably from about 0.2 to about 5.0 wt. %, more preferably from about 0.5 to about 2.0 wt. %, relative to the total weight of the cosmetic agent.

Below are the most preferred embodiments (A)-(E) of the cosmetic agent as contemplated herein, the cosmetic agent preferably being present as an oil-in-water emulsion:

### (A):

Cosmetic agent for coloring keratin fibers, contained in a cosmetically tolerated carrier—relative to its total weight—

a) from about 0.005 to about 25 wt. % of at least one of the oxidative dye precursors described above and/or partially oxidizing dyes
b) from about 0.0001 to about 20 wt. % of at least one of the silicone block copolymers described above
c) from about 0.0001 to about 10 wt. % of at least one of the phospholipids described above
d) from about 0 to about 45 wt. % of at least one of the alcohols and/or partial esters described above
e) from about 0 to about 5.0 wt. % of at least one of the thickening agents or gelling agents described above
f) from about 0 to about 15 wt. % of at least one of the alkalizing agents described above (B):

Cosmetic agent for coloring keratin fibers, contained in a cosmetically tolerated carrier—relative to its total weight—

a) from about 0.005 to about 25 wt. %, preferably from about 0.05 to about 20 wt. %, more particularly from about 0.1 to about 15 wt. % of at least one of the oxidative dye precursors and/or partially-oxidizing dyes described above
b) from about 0.0001 to about 20 wt. %, preferably from about 0.0005 to about 10 wt. %, preferably from about 0.005 to about 8.0 wt. %, more preferably from about 0.01 to about 5.0 wt. %, most preferably from about 0.1 to about 1.0 wt. % of at least one silicone block copolymer, containing at least one structural unit of the formula (I) and at least one structural unit of the formula (II) and at least one structural unit of the formula (III)

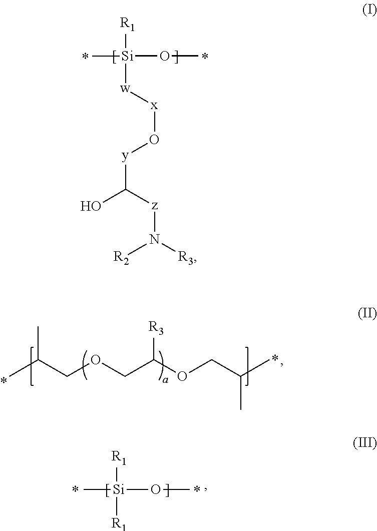

wherein
w denotes a direct bond or a heteroatom, selected from the group of NH, O or S,
x, y and z each independently denote a substituted or unsubstituted C₁-C₈-alkylene group,
R₁ denotes a substituted or unsubstituted C₁-C₄-alkyl radical,
R₂ and R₃ each independently denote a substituted or unsubstituted methyl-, ethyl-, n-propyl-, isopropyl-, n-butyl-, sec-butyl, isobutyl- or tert-butyl radical,
R₃ denotes hydrogen or a C₁-C₄-alkyl radical and
a denotes an integer between 1 and 30,
c) from about 0.0001 to about 10 wt. %, preferably from about 0.0005 to about 5.0 wt. %, preferably from about 0.005 to about 2.0 wt. %, more preferably from about 0.001 to about 1.0 wt., most preferably from about 0.05 to about 0.5 wt. % at least one phospholipid of the formula (Phos-1)

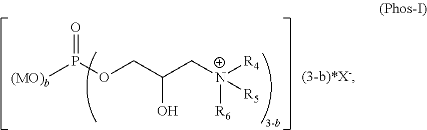

wherein
R₄ and R₆ each independently denote a C₁-C₄-alkyl radical, a C₁-C₄-hydroxyalkyl radical or a carboxyalkyl radical of the formula *—(CH2)_{c}COOM, wherein c means integers from 1 to 3 and M means hydrogen or a physiologically tolerated cation,
R₅ denotes a C₈-C₂₂-alkyl radical, a C₈-C₂₂-alkyl radical, a C₈-C₂₂-hydroxyalkyl radical or an acylaminoalkyl radical of the formula R⁷C(O)NH(CₘH₂ₘ)—*, wherein R⁷ means a linear C₈-C₂₂-acyl radical and m means an integer from 2 to 3,
b denotes integers from 0 to 3,
M denotes a physiologically tolerated cation, and
X⁻ denotes a physiologically tolerated anion
d) 0 to about 45 wt. %, preferably from about 5.0 to about 40 wt. %, preferably from about 10 to about 35 wt. %, more particularly from about 12 to about 30 wt. % 2-octyldodecanol and/or cetearyl alcohol and/or mixtures from glycerinmonostearate, glycerinmonopalmitate, glycerindistearate and glycerindipalmitate,
e) 0 to about 5.0 wt. %, preferably from about 0.001 to about 1.0 wt. %, more preferably from about 0.005 to about 0.5 wt. %, most preferably from about 0.01 to about 0.1 wt. % xanthan,
f) 0 to about 15 wt. %, preferably from about 0.5 to about 10 wt. %, more particularly from about 3.5 to about 7.5 wt. % monoethanolamine and/or ammoniac.

### (C):

Cosmetic agent for coloring keratin fibers, contained in a cosmetically tolerated carrier—relative to its total weight—

a) from about 0.1 to about 15 wt. % of at least one of the oxidative dye precursors described above
and/or partially oxidizing dyes
b) from about 0.1 to about 1.0 wt. % of at least one silicone block copolymer, containing at least one structural unit of the formula (I) and at least one structural unit of the formula (II) and at least one structural unit of the formula (III)

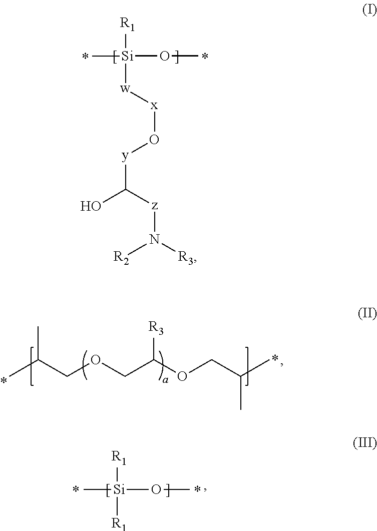

wherein
w denotes a direct bond or a heteroatom, selected from the group of NH, O or S,
x, y and z independently denote a substituted or unsubstituted C₁-C₈-alkylene group,
R₁ denotes a substituted or unsubstituted C₁-C₄-alkyl radical,
R₂ and R₃ independently denote a substituted or unsubstituted methyl-, ethyl-, n-propyl-, isopropyl-, n-butyl-, sec-butyl, isobutyl- or tert-butyl radical,
R₃ denotes hydrogen or a C₁-C₄ alky radical, and
a denotes an integer between 1 and 30.
c) from about 0.05 to about 0.5 wt. % of at least one phospholipid of the formula (Phos-1)

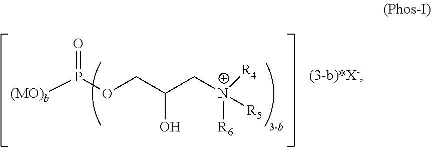

wherein
R₄ and R₆ independently denote a C₁-C₄-alkyl radical, a C₁-C₄-hydroxyalkyl radical or a carboxyalkyl radical of the formula *—(CH2)_{c}COOM, wherein c means integers from 1 to 3 and M means hydrogen or a physiologically tolerated cation,
R₅ denotes a C₈-C₂₂-alkyl radical, a C₈-C₂₂-alkyl radical, a C₈-C₂₂-hydroxyalkyl radical or an acylaminoalkyl radical of the formula R⁷C(O)NH(CₘH₂ₘ)—*, wherein R₇ means a linear C₈-C₂₂-acyl radical and m means an integer from 2 to 3,
b denotes integers from 0 to 3,
M denotes a physiologically tolerated cation, and
X⁻ denotes a physiologically tolerated anion
d) 0 to about 30 wt. % 2-octyldodecanol and/or cetearyl alcohol and/or mixtures of glycerinmonostearate, glycerinmonopalmitate, glycerindistearate and glycerindipalmitate,
e) 0 to about 0.1 wt. % xanthan,
f) 0 to about 7.5 wt. % monoethanolamine and/or ammoniac.

### (D):

Cosmetic agent for coloring keratin fibers, contained in a cosmetically tolerated carrier—relative to its total weight—

a) from about 0.1 to about 15 wt. % of at least one of the oxidative dye precursors described above
and/or partially oxidizing dyes
b) from about 0.1 to about 1.0 wt. % of at least one silicone block copolymer of the formula (IV)

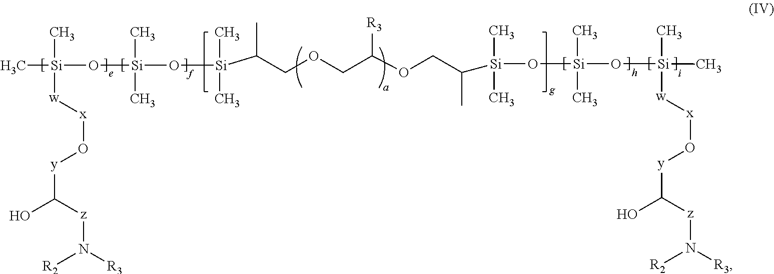

wherein
w denotes a direct bond or an oxygen atom,
x denotes a propyl group
y and z each independently denote a methyl group,
R₂ and R₃ each independently denote a 2-hydroxypropyl radical,
R₃ denotes hydrogen, and
a denotes integers from 4 to 25, preferably from 6 to 20, more preferably from 8 to 18, and most preferably from 12 to 16,
c) from about 0.05 to about 0.5 wt. % of at least one phospholipid of the formula (Phos-Ia)

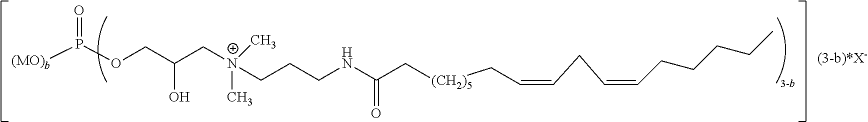

(Phos-1a), wherein
M denotes a sodium ion
X⁻ denotes a chloride ion, and
b denotes the integer 0 or 1
d) 0 to about 30 wt. % 2-octyldodecanol and/or cetearyl alcohol and/or mixtures of glycerinmonostearate, glycerinmonopalmitate, glycerindistearate and glycerindipalmitate,
e) 0 to about 0.1 wt. % xanthan,
f) 0 to about 7.5 wt. % monoethanolamine and/or ammoniac.

### (E):

Cosmetic agent for coloring keratin fibers, contained in a cosmetically tolerated carrier—relative to its total weight—

a) from about 0.1 to about 15 wt. % of at least one of the oxidative dye precursors described above
and/or partially oxidizing dyes
b) from about 0.1 to about 1.0 wt. % of at least one silicone block copolymer of the formula (IV)

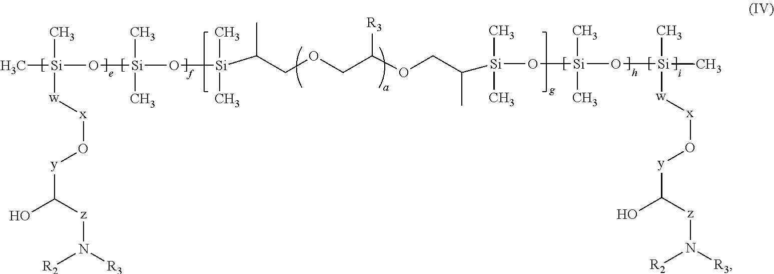

wherein
w denotes a direct bond or an oxygen atom,
x denotes a propyl group,
y and z each independently denote a methyl group,
R₂ and R₃ each independently denote a 2-hydroxypropyl radical,
R₃ denotes hydrogen, and
a denotes integers from 4 to 25, preferably from 6 to 20, more preferably from 8 to 18, and most preferably from 12 to 16,
c) from about 0.05 to about 0.5 wt. % of at least one phospholipid of the formula (Phos-Ia)

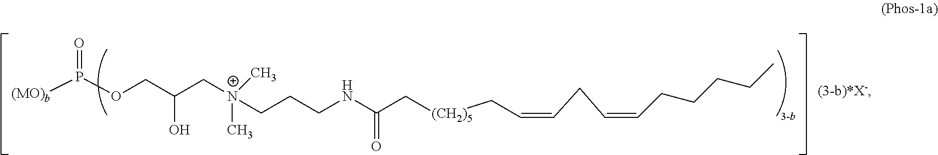

wherein
M denotes a sodium ion
X⁻ denotes a chloride ion, and
b denotes the integer 0 or 1
d) from about 15 to about 30 wt. % 2-octyldodecanol and/or cetearyl alcohol and/or mixtures of glycerinmonostearate, glycerinmonopalmitate, glycerindistearate and glycerindipalmitate,
e) from about 0.01 to about 0.1 wt. % xanthan,
f) from about 3.5 to about 7.5 wt. % monoethanolamine and/or ammoniac.

The aforementioned most preferred embodiments (A)-(E) of the cosmetic agent as contemplated herein are exemplified by an outstanding colorfulness of the shades that can be achieved by employing these agents.

Oxidative dye compositions can also be produced immediately prior to application from two or more separately packaged compositions. This is particularly useful for separating incompatible ingredients in order to prevent a premature reaction. A separation into multi component systems is preferred in particular wherever the ingredients are expected to be or suspected of being incompatible. In such cases, the oxidative dye composition is produced by the consumer by mixing immediately prior to application. According to the present disclosure, this procedure with oxidative dyes, wherein the cosmetic agent as contemplated herein is first separated from an oxidant preparation, containing at least one oxidant, is most preferred.

A further subject matter of the present disclosure is therefore a kit-of-parts, comprising—extensively packaged separately from one another—

a) at least one container (C1), containing a cosmetic agent as contemplated herein, and
b) at least one container (C2), containing an oxidant agent preparation, which contains in turn in a cosmetically tolerated carrier at least one oxidant and at least one acid.

The oxidative dye, which can be produced from the kit-of-parts as contemplated herein, and which contains at least one special silicone block copolymer and at least one phospholipid, leads to enhanced colorfulness of the keratin fibers that are colored by employing said dyes. In order to achieve the same colorfulness as conventional oxidative dyes without the aforementioned combination of silicone block copolymers and phospholipids, the quantity of dyes for the oxidative dyes produced from the kit-of-parts as contemplated herein can be significantly reduced.

According to the present disclosure, the term “container” is an envelope, which is present in the form of a possibly reclosable bottle, tube, jar, bag, sachet or similar envelopes. As contemplated herein, there are no restrictions with respect to the envelope material. However, envelopes from glass or plastic are preferred.

The oxidants according to the present disclosure are different to atmospheric oxygen. Hydrogen peroxide, as well as the solid sediments for organic and inorganic compounds thereof, can be used as oxidants. As contemplated herein, the sediments for urea, melamine, polyvinyl-pyrrolidinon, as well as sodium borate, can be used as solid sediments. Hydrogen peroxide and/or one of the solid sediments for organic or inorganic compounds thereof are the most preferred oxidants. As contemplated herein, the oxidant is therefore preferably selected from the group of persulphates, chlorites, hydrogen peroxide and sediments of hydrogen peroxide for urea, melamine, as well as sodium borate, more particularly hydrogen peroxide.

A particularly preferable embodiment of the present disclosure is therefore exemplified in that, as an oxidant, hydrogen peroxide is contained in a total quantity of from about 0.5 to about 15 wt. %, preferably from about 0.75 to about 10 wt. %, more preferably from about 1 to about 7.5 wt. %, even more preferably from 1.25 to about 7 wt. %, most preferably from about 1.5 to about 6.0 wt. %, relative to the total weight of the oxidant preparation. The calculation of the total quantity refers to about 100% H₂O₂.

The oxidant preparations can also contain water in a total quantity of from about 40 to about 98 wt. %, more particularly from about 65 to about 85 wt. %, relative to the total weight of the oxidant preparation.

According to a preferred embodiment of the present disclosure, the oxidant preparations also contain at least one linear saturated alkanol with 12 to 30 carbon atoms, more particularly with 16 to 22 carbon atoms, in a total quantity of 0.1 to 10 wt. %, preferably 0.5 to 5.0 wt. %, more particularly 1.0 to 4.0 wt. %, relative to the total weight of the oxidant preparation. The preference is for cetyl alcohol, stearyl alcohol, arachidyl alcohol, behenyl alcohol and lanolin alcohol or mixtures of said alcohols, as available for the large-scale hydration of plant and animal fatty acids, as well as mixtures of said alcohols. The mixture cetearyl alcohol is most preferred.

In another preferred embodiment of the present disclosure, the oxidant preparations contain at least one ethoxylated non-ionic tenside, preferably selected from tensides with the INCI designation ceteth-12, steareth-12, ceteareth-12, ceteth-20, steareth-20, ceteareth-20, ceteth-30, steareth-30, ceteareth-30, oleth-30, ceteareth-50, PEG-40 hydrogenated castor oil and PEG-60 hydrogenated castor oil, as well as mixtures of said substances, most preferably selected from ceteth-20, steareth-20, ceteareth-20, ceteth-30, steareth-30 and ceteareth-30, in a total quantity of from about 0.1 to about 10 wt. %, preferably from about 0.5 to about 5.0 wt. %, more preferably from 1 to about 4.0 wt. %, relative to the total weight of the oxidant preparation.

According to the present disclosure, the oxidant preparations can also contain at least one ester from carboxylic acid with 10 to 20 carbon atoms and one linear or branched alcohol with 1 to 5 carbon atoms, more particularly isopropylmyristate, in a total quantity of from about 3.0 to about 25 wt. %, preferably from about 5.0 to about 20 wt. %, more preferably from about 8.0 to about 15 wt. %, relative to the total weight of the oxidant preparation.

According to a most preferred embodiment of the present disclosure, the oxidant preparations contain—relative to the total weight of the oxidant preparations—
at least one linear saturated alcanol with 12 to 30 carbon atoms in a total quantity of from about 0.1 to about 10 wt. %, preferably from about 0.5 to about 5.0 wt. %, more particularly from about 1.0 to about 4.0 wt. %, further
at least one ethoxylated non-ionic tenside, preferably selected from tensides with the INCI designation ceteth-12, steareth-12, ceteareth-12, ceteth-20, steareth20, ceteareth-20, ceteth-30, steareth-30, ceteareth-30, oleth-30, ceteareth-50, PEG-40 hydrogenated castor oil and PEG-60 hydrogenated castor oil, as well as mixtures of said substances, most preferably selected from ceteth-20, steareth-20, ceteareth-20, ceteth-30, steareth-30 and ceteareth-30, in a total quantity of from about 0.1 to about 10 wt. %, preferably from about 0.5 to about 5.0 wt. %, more preferably from about 1.0 to about 4.0 wt. %, as well as
at least one ester from a carboxylic acid with 10 to 20 carbon atoms and one linear or branched alcohol with 1 to 5 carbon atoms, preferably isopropylmyristate, in a total quantity of from about 3.0 to about 25 wt. %, preferably from about 5.0 to about 20 wt. %, more particularly from about 8.0 to about 15 wt. %.

The oxidant preparations as contemplated herein also contain at least one acid. Preferred acids are selected from dipicolinic acids, food acids, such as citric acid, acetic acid, malic acid, lactic acid and tartaric acid, diluted mineral acids such as hydrochloric acid, phosphoric acid, pyrophosphoric acid and sulphuric acid, as well as mixtures thereof. The oxidant preparations preferably have a pH value in the range of from about 2 to about 5, more particularly from about 3 to about 4.

To produce oxidative dye compositions from the kit-of-parts as contemplated herein, the cosmetic agent as contemplated herein is mixed in the container C₁ with the oxidant preparation in container C₂ or vice versa.

Moreover, it can be particularly advantages as contemplated herein for the kit-of-parts to have a further hair treatment agent, more particularly a conditioner preparation, in an additional container. This conditioner preparation contains, advantageously, at least one conditioning agent, selected from the group of cationic polymers, silicone derivatives and oils. Moreover, the kit-of-parts can comprise application aids, such as combs, brushes, applicators or brushes, personal protective clothing, more particularly disposable gloves, as well as instructions for use. An applicator is a wide brush, located at the stem end of which is a tip, which permits and simplifies the division of fiber bundles and/or hair strands from the total quantity of fibers.

With respect to the cosmetic agent as contemplated herein in container C₁ and the oxidant preparation in container C₂, the statements made about the cosmetic agents as contemplated herein apply mutatis mutandis.

A further subject matter of the present disclosure is a method for coloring keratin fibers, wherein the method comprises the following steps:

a) Provision of a cosmetic agent as contemplated herein (M1),
b) Provision of an oxidant preparation (M2), containing in a cosmetically tolerated carrier at least one oxidant and at least one acid,
c) Mixing of the cosmetic agent (M1) with the oxidant preparation (M2),
d) Application of the mixture obtained in Step c) onto the keratin fibers and leaving said mixture for a period of from about 10 to about 60 minutes, preferably from about 20 to about 45 minutes at room temperature and/or at least about 30° C. on the keratin fibers,
e) rinsing the keratin fibers with water and/or a cleansing composition for from about 1 to about 5 minutes, and
f) where applicable applying a post-treatment agent to the keratin fibers and rinsing after a period from about 1 to about 10 minutes.

The method for coloring keratin fibers as contemplated herein using a combination of silicone block copolymers and phospholipids results in an enhanced colorfulness of shades compared to methods in which the aforementioned combination is not used. As a result, the same colorfulness as that achieved with methods of the prior art, in which this combination is not used, can be achieved with a smaller quantity of dyes.

Room temperature according to the present disclosure means the ambient temperature that prevails without the effect of external heat and amounts to preferably from about 10 to about 39 degrees C. The effect of the coloring or lightening preparation can be increased through an external heat supply, by employing a hot hood for example. The preferred exposure time of the coloring and/or lightening preparation on the keratin fibers is from about 10 to about 60 min, preferably from about 20 to about 45 min. At the end of the exposure period, the remaining dye is washed out of the keratin fibers by employing a cleansing preparation, which preferably contains at least one cationic and/or anionic and/or non-ionic tenside. The process is repeated with a further agent as necessary. After the washing out step, the keratin fibers are rinsed, as necessary, with a post-treatment agent, a conditioning agent for example, and then dried with a hand towel or a hot air blower. The dye preparation is normally applied by hand by the user. Personal protective clothing is preferably worn in the process, more particularly protective gloves, preferably from plastic or latest for one-time use (disposable gloves), as well as an apron. However, the dyes can also be applied to the keratin fibers by employing an application aid.

With respect to the cosmetic agent (M1), the oxidant preparation (M2) and other preferred embodiments of the method as contemplated herein, the statements made about the cosmetic means and the kit-of-parts as contemplated herein apply mutatis mutandis.

Moreover, a further subject matter of the present disclosure is the use of a cosmetic agent as contemplated herein for increasing the colorfulness of shades. The use of a combination of silicone block copolymers and phospholipids results in an enhanced colorfulness of shades. As a result, the quantity of dyes used in the cosmetic agent as contemplated herein can be significantly less than agents of the prior art without such combination, in order to achieve an identical colorfulness as with oxidative dyes of the prior art.

With respect to the preferred embodiments of the use as contemplated herein, the statements made about the cosmetic agent as contemplated herein apply mutatis mutandis.

Finally, a further subject matter of the present disclosure is the use of a kit-of-parts as contemplated herein for producing a cosmetic agent for coloring keratin fibers with increased colorfulness of shades. As already stated, the use of a combination from silicon block copolymers and phospholipids results in an increased colorfulness of shades, which permits the quantity of dyes to be reduced.

With respect to the preferred embodiments of the use as contemplated herein, the statements made about the cosmetic agent as contemplated herein apply mutatis mutandis.

The present disclosure is outlined in particular by the following items:
1. Cosmetic agent for coloring keratin fibers, comprising in a cosmetically tolerant carrier
a) at least one compound, selected from the group of oxidative dye precursors, partially-oxidative dyes and the mixtures thereof,
b) at least one silicone block copolymer, containing at least one structural unit of the formula (I) and at least one structural unit of the formula (II) and at least one structural unit of the formula (III)

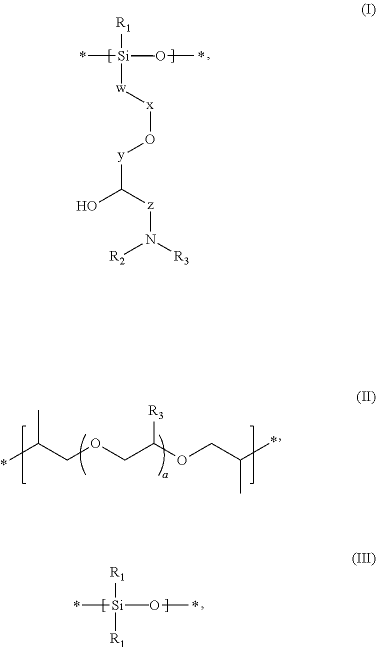

wherein
w denotes a direct bond or a heteroatom, selected from the group of NH, O or S,
x, y and z each independently denote a substituted or unsubstituted C₁-C₈-alkylene group,
R₁ denotes a substituted or unsubstituted C₁-C₄-alkyl radical,
R₂ and R₃ each independently denote a substituted or unsubstituted methyl-, ethyl-, n-propyl-, isopropyl-, n-butyl-, sec-butyl, isobutyl- or tert-butyl radical,
R₃ denotes hydrogen or a C₁-C₄-alkyl radical and
a denotes an integer between 1 and 30,
and
c) at least one phospholipid of the formula (Phos-1)

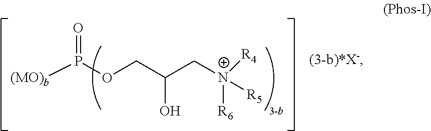

wherein
R₄ and R₆ independently denote a C₁-C₄-alkyl radical, a C₁-C₄-hydroxyalkyl radical or a carboxyalkyl radical of the formula *—(CH2)_{c}COOM, wherein c means integers from 1 to 3 and M means hydrogen or a physiologically tolerated cation,
R₅ denotes a C₈-C₂₂-alkyl radical, a C₈-C₂₂-alkyl radical, a
C₈-C₂₂-hydroxyalkyl radical or an acylaminoalkyl radical of the formula
R⁷C(O)NH(CₘH₂ₘ)—*, wherein R⁷ means a linear C₈-C₂₂-acyl radical and m means an integer from 2 to 3,
b denotes integers from about 0 to about 3,
M denotes a physiologically tolerated cation, and
X⁻ denotes a physiologically tolerated anion.
2. Cosmetic agent according to item 1, exemplified in that the cosmetic agent contains the at least one oxidative dye precursor in a total quantity of from about 0.005 to about 25 wt. %, preferably from about 0.05 to about 20 wt. %, more preferably from about 0.1 to about 15 wt. %.
3. Cosmetic agent according to one of the items 1 or 2, exemplified in that the cosmetic agent contains the at least one partially-oxidizing dye in a total quantity of from about 0.001 to about 10 wt. %, preferably from about 0.01 to about 8 wt. %, more preferably from about 0.1 to about 5 wt. %, and most preferably from about 0.5 to about 3 wt. %, relative to the total weight of the cosmetic agent.
4. Cosmetic agent according to one of the aforementioned items, exemplified in that in the formula (I), w denotes a direct bond or an oxygen atom, x denotes a C₂-C₄-alkyl group, y and z each denote independently a C₁-C₃-alkyl group, and R₁ and R₂ each independently denote a hydroxypropyl radical.
5. Cosmetic agent according to one of the aforementioned items, exemplified in that in the formula (II), the radical R₃ denotes a hydrogen atom and a denotes integers from 4 to 25, preferably from 6 to 20, preferably from 8 to 18, preferably from 12 to 16.
6. Cosmetic agent according to one of the aforementioned items, exemplified in that the silicone block copolymer b) contains at least one structural unit (Ia)

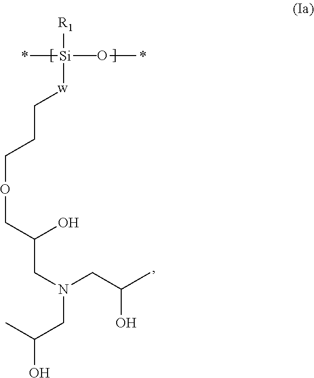

wherein
w denotes a direct bond or an oxygen atom.
7. Cosmetic agent according to one of the items, exemplified in that the cosmetic agent contains as the silicone block copolymer b) a silicone block polymer of the formula (IV)

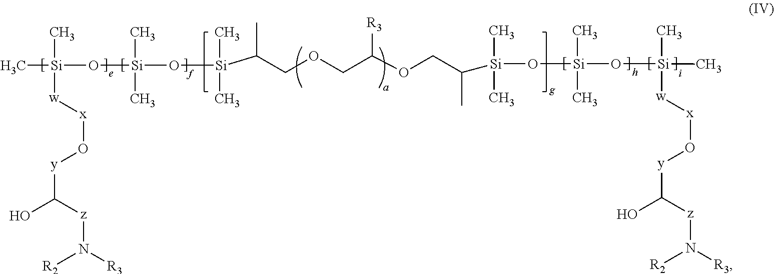

wherein
w denotes a direct bond or an oxygen atom.
x denotes a propyl group,
y and z each independently denote a methyl group,
R₂ and R₃ each independently denote a 2-hydroxypropyl radical,
R₃ denotes hydrogen, and
a denotes integers from about 4 to about 25, preferably from about 6 to about 20, more preferably from about 8 to about 18, most preferably from about 12 to about 16.
8. Cosmetic agent according to one of the aforementioned items, exemplified in that at least one silicone block copolymer b) is a silicone block copolymer from polyethylenglycol-bis(2-methyl-2-propen-1-yl)ether, 3-[3-[bis(2-hydroxypropyl)amino]-2-hydroxypropoxy]propyl-siloxane and dimetyhlsiloxane.
9. Cosmetic agent according to one of the aforementioned items, exemplified in that the cosmetic agent contains the at least one silicone block copolymer b) in a total quantity of from about 0.0001 to about 20 wt. %, preferably from about 0.0005 to about 10 wt.-%, more preferably from about 0.005 to about 8.0 wt. %, even more preferably from about 0.01 to about 5.0 wt. %, and most preferably from about 0.1 to about 1.0 wt. %, relative to the total weight of the cosmetic agent.
10. Cosmetic agent according to one of the aforementioned items, exemplified in that in the formula (Phos-1), radical R₅ denotes an acylaminoalkyl radical of the formula R⁷C(O)NH(CₘH₂ₘ)—*, wherein R⁷ means a linear C₈-C₂₂-acyl radical and m denotes an integer from about 2 to about 3, radicals R₄ and R₆, each independently denote a C₁-C₂ alkyl radical, b denotes the integer about 0 or about 1, M denotes an alkali metal, more particularly lithium, potassium or sodium, and x⁻ denotes a halide ion, more particularly chloride, bromide or iodide.
11. Cosmetic agent according to one of the aforementioned items, exemplified in that the cosmetic agent contains as the phospholipide c) a phospholipid of the formula (Phos-1a)

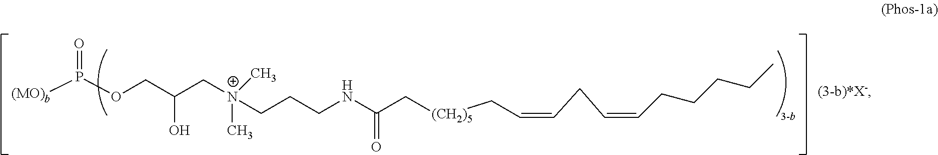

wherein
M denotes a sodium ion
X⁻ denotes a chloride ion, and
b denotes the integer about 0 or about 1.
12. Cosmetic agent according to one of the aforementioned items, exemplified in that the cosmetic agent contains the at least one phospholipid c) in a total quantity of from about 0.0001 to about 10 wt. %, preferably from about 0.0005 to about 5.0 wt.-%, more preferably from about 0.005 to about 2.0 wt. %, even more preferably from about 0.001 to about 1.0 wt. %, and most preferably from about 0.05 to about 0.5 wt. %, relative to the total weight of the cosmetic agent.
13. Kit-of-parts, comprising separately packed
a) at least one container (C₁), containing a cosmetic agent according to one of the claims 1 to 19, and
b) at least one container (C₂), containing an oxidizing agent preparation, which contains in turn in a cosmetically tolerated carrier at least one oxidant.
14. Method for coloring keratin fibers, wherein the method comprises the following steps:
a) Provision of a cosmetic agent (M1) according to one of the items 1 to 20,
b) Provision of an oxidant preparation (M2), containing in a cosmetically tolerated carrier at least one oxidant,
c) Mixing of the cosmetic agent (M1) with the oxidant preparation (M2),
d) Application of the mixture obtained in Step c) onto the keratin fibers and leaving said mixture for a period of from about 10 to about 60 minutes, preferably from about 20 to about 45 minutes at room temperature and/or at least about 45° C. on the keratin fibers,
e) rinsing the keratin fibers with water and/or a cleansing composition for from about 1 to about 5 minutes, and
f) where applicable applying a post-treatment agent to the keratin fibers and rinsing off after a period of from about 1 to about 10 minutes.
15. Use of a cosmetic agent according to one of the items 1 to 19 to improve the colorfulness of the shades.
The examples below explain, but do not limit preferred embodiments of the present disclosure.

### EXAMPLES

### 1. Recipes

Compositions of the used cosmetic agents (oil-in-water emulsions, all quantities in wt. %). The silicone block copolymer used in the formulations below is preferably a silicone block copolymer of the formula (IV). Moreover, a phospholipid of the formula (Phos-1a) is preferably used as the phospholipid.

| Raw material | V2 | E1* | V1 |
|---|---|---|---|
| Xanthan gum | 0.1 | 0.1 | 0.1 |
| 2-Octyldodecanol | 2.3 | 2.3 | 2.3 |
| Lanette N ^{a)} | 7.0 | 7.0 | 7.0 |
| Cetearyl alcohol | 2.0 | 2.0 | 2.0 |
| Glycerl stearate | 3.0 | 3.0 | 3.0 |
| Glycerol 99.5% | 1.0 | 1.0 | 1.0 |
| Coconut amidopropylbetaine, 40% ig | 1.3 | 1.3 | 1.3 |
| Monoethanolamin | 4.4 | 4.4 | 4.4 |
| Ammonia, 25% ig | 1.0 | 1.0 | 1.0 |
| Sodium sulfite, anhydrous | 0.3 | 0.3 | 0.3 |
| 1-Hydroxyethyl-4,5-diaminopyrazolsulfate | 1.5 | 1.5 | 1.5 |
| 4-Amino-3-methylphenol | 0.1 | 0.1 | 0.1 |
| p-Amino-o-cresol | 0.2 | 0.2 | 0.2 |
| m-Aminophenol | 0.6 | 0.6 | 0.6 |
| Silicone block copolymer ** | 1.1 | — | 1.1 |
| Phospholipid ** | — | 0.15 | 0.15 |
| Water, fully-demineralized | ad | ad | ad |
| | 100.00 | 100.00 | 100.00 |

*as contemplated herein
** Active substance
^{a)} INCI designation: Cetearyl alcohol, Sodium cetearyl sulfate (BASF)

The fat basis was fused together at 80° C. and dispersed with a portion of the water quantity. The remaining recipe constituents were then gradually incorporated by agitation. Water was then added to 100 wt. % and the formulation was agitated cold. Recipes V1 and V2 are not-invention comparison recipes without silicone block copolymers and/or phospholipids. Recipe E1 is an example as contemplated herein.

Oxidant preparation O1 (all quantities in wt. %)

| Raw material | O1 |
|---|---|
| Di-sodium pyrophosphate | 0.1 |
| Dipicolinic acid | 0.1 |
| Potassium hydroxide 50% | 0.3 |
| 1-hydroxyethane-1,1-diphosphonic acid 60% | 0.4 |
| Fatty alcohol sulphate, sodium salt, C16-C18 | 0.3 |
| Eumulgin RO 40 b) | 0.6 |
| Cetearyl alcohol | 3.6 |
| Ceteareth-20 | 0.5 |
| Beeswax | 0.3 |
| lsopropylmyristat | 10 |
| Hydrogen peroxide 50% | 11 |
| Water, fully-demineralized | ad 100 |

b) INCI designation: PEG-40 Castor 011 (BASF)

2. Enhanced colorfulness through the addition of a mixture of silicone block copolymers and phospholipids. To produce oxidative dyes for determine colorfulness, the cosmetic agents V1, V2 and E1 were each mixed with the aforementioned oxidant preparation in the weight ratio of 1:1.

The oxidative dyes thus produced were each applied in defined amounts (4 g of oxidative dye per 1 g of yak hair) on yak hair (12 strands per oxidant) and left for an exposure period of 30 minutes at 32° C. The remaining agents were then rinsed out of the hair strands with luke-warm water for 2 minutes each, the strands first being dried with a hand towel and then blown dry.

All strands were measured with a colorimeter from Datacolor, type Spectraflash 450. An indicator of the colorfulness of shades is the chroma. The higher the dC value, the higher the colorfulness of the shade. The table below shows the dC values for the shades using cosmetic agents V1, V2 and E1. The shades created by the cosmetic agent E1 as contemplated herein, which contain a combination of a silicone block copolymer and a phospholipid, have better colorfulness than the shade created with the silicon block copolymer (V1) and the phospholipid (V2).

| Oxidative dye | dC |
|---|---|
| V1 + 01 (1:1) | 31.3 |
| V2 + 01 (1:1) | 31.7 |
| E1 + 01 (1:1) | 33.2 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic agent for coloring keratin fibers, comprising in a cosmetically tolerant carrier:
a) at least one compound, selected from the group of oxidative dye precursors, partially-oxidative dyes and the mixtures thereof;
b) at least one silicone block copolymer, comprising at least one structural unit of the formula (I) and at least one structural unit of the formula (II) and at least one structural unit of the formula (III) 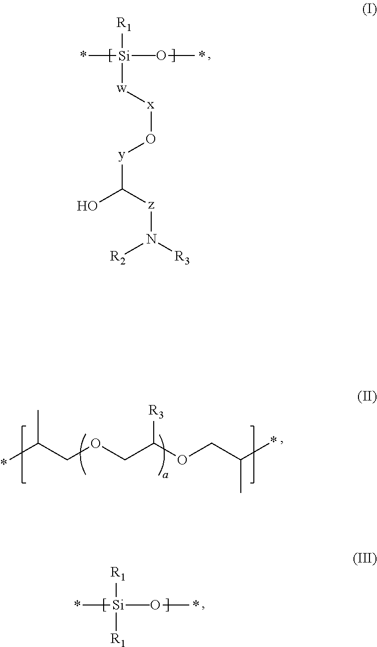
wherein
w denotes a direct bond or a heteroatom, selected from the group of NH, O or S,
x, y and z each independently denote a substituted or unsubstituted C₁-C₈-alkylene group,
R₁ denotes a substituted or unsubstituted C₁-C₄-alkyl radical,
R₂ and R₃ each independently denote a substituted or unsubstituted methyl-, ethyl-, n-propyl-, isopropyl-, n-butyl-, sec-butyl, isobutyl- or tert-butyl radical,
R₃ denotes hydrogen or a C₁-C₄-alkyl radical, and
a denotes integers from 1 to 30; and
c) at least one phospholipid of the formula (Phos-1) 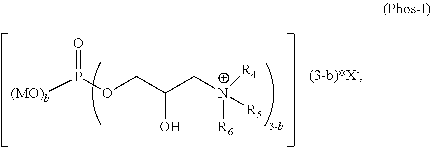
wherein
R₄ and R₆ each independently denote a C₁-C₄-alkyl radical, a C₁-C₄-hydroxyalkyl radical or a carboxyalkyl radical of the formula *—(CH2)_{c}COOM, wherein c means integers from 1 to 3 and M means hydrogen or a physiologically tolerated cation,
R₅ denotes a C₈-C₂₂-alkyl radical, a C₈-C₂₂-alkyl radical, a C₈-C₂₂-hydroxyalkyl radical or an acylaminoalkyl radical of the formula R⁷C(O)NH(CₘH₂ₘ)—*, wherein R⁷ means a linear C₈-C₂₂-acyl radical and m means an integer from 2 to 3,
B denotes integers from 0 to 3,
M denotes a physiologically tolerated cation, and
X⁻ denotes a physiologically tolerated anion.

2. Cosmetic agent according to claim 1, wherein the silicone block copolymer b) comprises at least one structural unit (Ia) 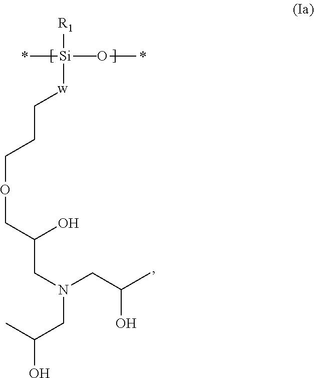
wherein w denotes a direct bond or an oxygen atom.

3. Cosmetic agent according to claim 1, wherein the cosmetic agent comprises as the silicone block copolymer b) a silicone block polymer of the formula (IV) 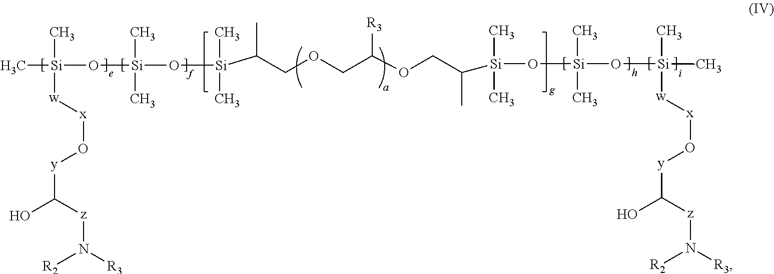
wherein
w denotes a direct bond or an oxygen atom,
x denotes a propyl group,
y and z each independently denote a methyl group,
R₂ and R₃ each independently denote a 2-hydroxypropyl radical,
R₃ denotes hydrogen, and
a denotes integers from 4 to 25.

4. Cosmetic agent according to claim 1, wherein the cosmetic agent comprises the at least one silicone block copolymer b) in a total quantity of from about 0.0001 to about 20 wt. %, relative to the total weight of the cosmetic agent.

5. Cosmetic agent according to claim 1, wherein the cosmetic agent comprises as the 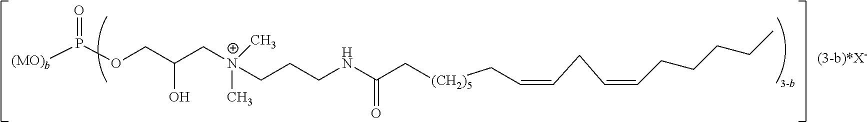
phospholipide c) a phospholipid of the formula (Phos-1a)
(Phos-1a),
wherein
M denotes a sodium ion
X⁻ denotes a chloride ion, and
b denotes the integer about 0 or about 1.

6. Cosmetic agent according to claim 1, wherein the cosmetic agent comprises the at least one phospholipid c) in a total quantity of from about 0.0001 to about 10 wt. %, relative to the total weight of the cosmetic agent.

7. Kit-of-parts, comprising separately packed:
a) at least one container (C1), comprising a cosmetic agent according to claim 1, and
b) at least one container (C2), comprising an oxidizing agent preparation, which comprises in turn in a cosmetically tolerated carrier at least one oxidant.

8. Method for coloring keratin fibers, wherein the method comprises the following steps:
a) Provision of a cosmetic agent (M1) according to claim 1,
b) Provision of an oxidant preparation (M2), comprising in a cosmetically tolerated carrier at least one oxidant,
c) Mixing of the cosmetic agent (M1) with the oxidant preparation (M2),
d) Application of the mixture obtained in Step c) onto the keratin fibers and leaving said mixture for a period of from about 10 to about 60 minutes at least 45° C. on the keratin fibers,
e) rinsing the keratin fibers with water and/or a cleansing composition for from about 1 to about 5 minutes, and
f) where applicable applying a post-treatment agent to the keratin fibers and rinsing after a period from about 1 to about 10 minutes.

9. Cosmetic agent according to claim 1, wherein the cosmetic agent is utilized to improve the colorfulness of the shades.

10. (canceled)

11. Cosmetic agent according to claim 4, wherein the cosmetic agent comprises the at least one silicone block copolymer b) in a total quantity of from about 0.0005 to about 10 wt. %, relative to the total weight of the cosmetic agent.

12. Cosmetic agent according to claim 4, wherein the cosmetic agent comprises the at least one silicone block copolymer b) in a total quantity of from about 0.005 to about 8.0 wt. %, relative to the total weight of the cosmetic agent.

13. Cosmetic agent according to claim 4, wherein the cosmetic agent comprises the at least one silicone block copolymer b) in a total quantity of from about 0.01 to about 5.0 wt. %, relative to the total weight of the cosmetic agent.

14. Cosmetic agent according to claim 4, wherein the cosmetic agent comprises the at least one silicone block copolymer b) in a total quantity of from about 0.1 to about 1.0 wt. %, relative to the total weight of the cosmetic agent.

15. Cosmetic agent according to claim 6, wherein the cosmetic agent comprises the at least one phospholipid c) in a total quantity of from about 0.0005 to about 5.0 wt. %, relative to the total weight of the cosmetic agent.

16. Cosmetic agent according to claim 6, wherein the cosmetic agent comprises the at least one phospholipid c) in a total quantity of from about 0.005 to about 2.0 wt. %, relative to the total weight of the cosmetic agent.

17. Cosmetic agent according to claim 6, wherein the cosmetic agent comprises the at least one phospholipid c) in a total quantity of from about 0.001 to about 1.0 wt. %, relative to the total weight of the cosmetic agent.

18. Cosmetic agent according to claim 6, wherein the cosmetic agent comprises the at least one phospholipid c) in a total quantity of from about 0.05 to about 0.5 wt. %, relative to the total weight of the cosmetic agent.

19. Cosmetic agent according to claim 3, wherein a denotes integers from 6 to 20.

20. Cosmetic agent according to claim 3, wherein a denotes integers from 8 to 18.

21. Cosmetic agent according to claim 3, wherein a denotes integers from 12 to 16.
